(19) United States
(12) Patent Application Publication
(10) Pub. No.: US 2004/0024341 A1
(43) Pub. Date: Feb. 5, 2004

(54) Protective device

(76) Inventor: Scott Jacobs, Lakewood, CO (US)

Correspondence Address: John S. Munday, Esquire, LAW OFFICES OF JOHN S. MUNDAY, P.O. BOX 423, Isanti, MN, 55040, US

(21) Appl. No.: 10/210,269

(22) Filed: Jul. 31, 2002

(51) Int. Cl. A61F 13/00; A41D 13/00; A41D 27/26

(52) U.S. Cl. 602/72; 2/466

(57) ABSTRACT

An athletic cup having a shape generally designed to have the narrow portion fit between the legs of the user while the upper or wider portion extends up over the area of the body being protected. The cup has a larger primary portion formed from a resilient thermoplastic composition having a quantity of gas pockets dispersed therein formed by heating of a mixture of a blowing agent and a resilient thermoplastic material. The cup further includes a hard portion bonded to said primary portion and forming a cage like outer segment having a high degree of rigidity. The primary portion also includes a peripheral lip having a degree of flexibility for contact with the user to provide additional cushioning. In a preferred embodiment the primary portion extends into interstices in said hard portion.

### FIELD OF THE INVENTION

The present invention relates to protective cups for use by males in sports events such as baseball and the like, for insertion in a athletic supporter for protection of the user. More particularly, the protective device is formed from a composition having a quantity of gas pockets dispersed therein by heating a mixture of a thermoplastic compound and a blowing agent, and specifically to an improved protective cup formed from the composition to provide greater protection against shocks or blows.

### BACKGROUND OF THE INVENTION

Current athletic cups are sometimes formed from hard thermoplastic materials of sufficient strength to resist the impact of, for example, a baseball traveling at high speed. Major league pitchers throw a baseball at speed up to 100 miles per hour and batted balls have a much greater velocity. If such an object would strike a male in the genital area by misadventure, intense pain and severe damage might occur.

The conventional athletic cup, being of hard material also included a slightly softer rim portion which is intended to ease the force of the cup as it is driven into the ball player's body. While these cups are designed to protect a user against physical shock or blows, the impact of the device itself on the body can cause collateral injury. While the direct genital area is protected, the surrounding tissue and muscle can be severely bruised, even enough to prevent the player from continuing in the game. The design of commercially available devices during normal use absorb, attenuate, or deflect such blows to decrease the resultant transmitted force in an attempt to decrease or minimize injury to the user. They are not satisfactory in preventing collateral injury, nor are they totally comfortable to wear due to the rigid construction.

Accordingly, it is an object of the present invention to provide a resilient composition for use as an athletic cup for use by ballplayers and the like.

Another object is to employ a cushioned yet strong material to form the athletic cup, such as those polymers having gas pockets therein to increase attenuation and dampening of shocks or blows applied thereto.

Yet another object of the present invention is to provide a unitary athletic cup which is flexible to a limited degree, to thereby provide additional dissipation of the impact and spread the impact over a larger part of the device, thus not concentrating the force only on the perimeter of the cup.

Still another object of the present invention is to provide a one-piece athletic cup capable of slight flexibility on its sides to increase comfort when worn in athletic competition.

Other objects will appear hereinafter.

### SUMMARY OF THE INVENTION

It has now been discovered that the above and other objects of the present invention may be accomplished in the following manner. Specifically, the present invention provides an athletic cup made from a resilient material having a quantity of gas pockets dispersed therein and a rigid plastic cage supporting the resilient material to prevent collapse thereof. The resilient material which is the primary portion of the device, is formed from heating a mixture of a resilient thermoplastic material and a blowing agent. In the preferred embodiment the thermoplastic material is ethylene vinyl acetate (hereinafter EVA) and the blowing agent is p, p′-oxybis (benzenesulfonyl hydrazide). The rigid plastic cage is preferably formed from hard polymers and most preferably from acrylonitrile-butadiene-styrene copolymers, hereinafter (ABS) having a high degree of hardness as defined herein below.

An injection molded athletic cup is formed from the resilient material by mixing a predetermined quantity of blowing agent with a compatible thermoplastic material and then heating the mixture to a predetermined temperature range to form gas pockets in the resultant mixture. The heated mixture is then injection molded in a mold. The device is cooled and removed from the mold. A second mold produces the plastic cage portion and the two are mated to form the final product. Alternatively the protective device may be completed at the factory and sold as a completed product.

### BRIEF DESCRIPTION OF THE DRAWINGS

For a more complete understanding of the invention, reference is hereby made to the drawings, in which:

FIG. 1 is a perspective view of the preferred embodiment of the present invention;

FIG. 2 is a front elevational view of the device of FIG. 1;

FIG. 3 is a side elevational view of the device of FIG. 1;

FIG. 4 is a sectional view along the line 4-4 of the device of FIG. 1;

FIG. 5 is a sectional view along the line 5-5 of the device of FIG. 1; and

FIG. 6 is a sectional view along the line 6-6 of the device of FIG. 1.

### DETAILED DESCRIPTION OF THE PREFERRED EMBODIMENT

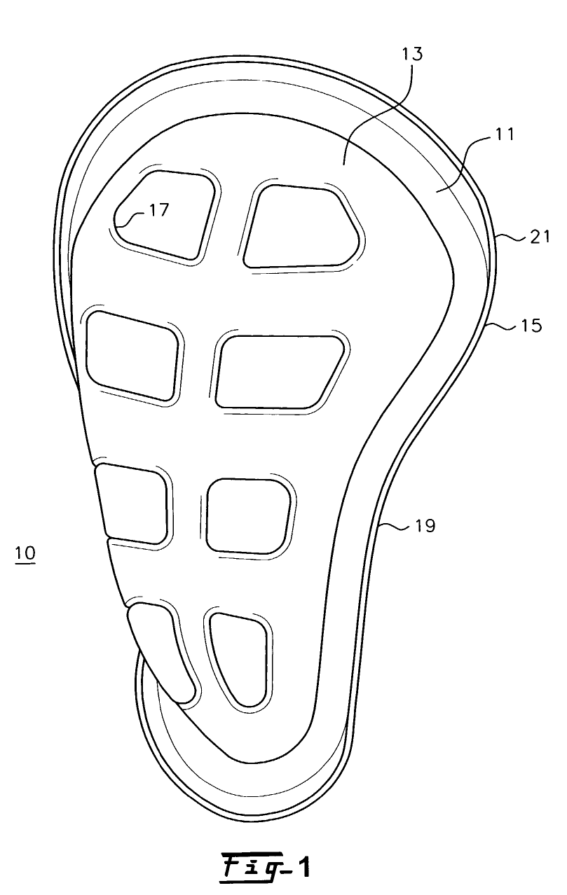

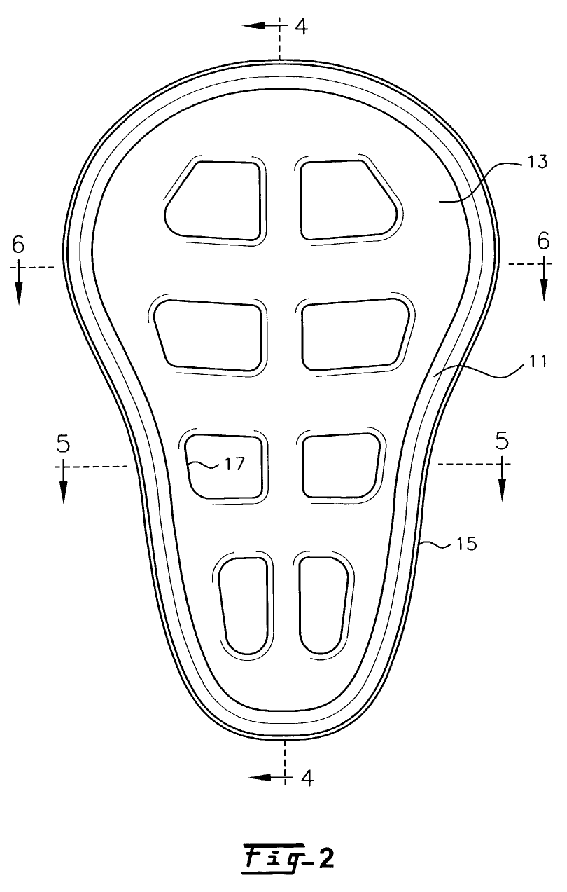

As shown in the drawings, an athletic cup in accordance with the invention, generally shown as 10, is formed into a primary portion 11, a hard portion 13, a peripheral lip 15, and interstices 17 in the hard portion. The combination of primary portion and hard portion has a shape generally designed to have the narrow portion 19 fit between the legs of the male user while the upper or wider portion 21 extends over the area of the body being protected. FIGS. 1-3 show the device in several views.

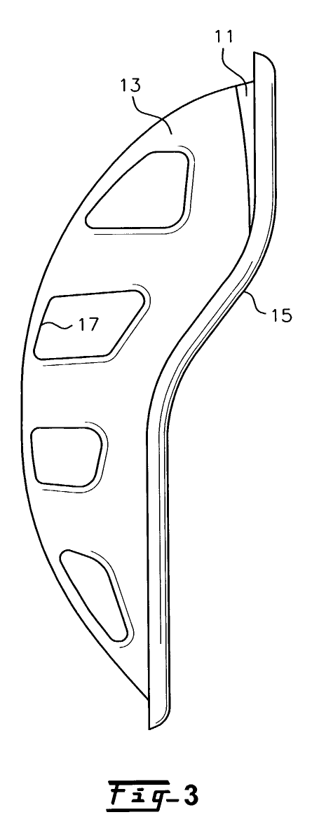

Primary portion 11 is formed from a resilient thermoplastic composition having a quantity of gas pockets 23 dispersed therein, As shown in FIGS. 4-6, gas pockets 23 are relatively small and are evenly dispersed throughout the entire primary portion 11. Gas pockets 23 are formed by the heating of a mixture of a blowing agent and a resilient thermoplastic material.

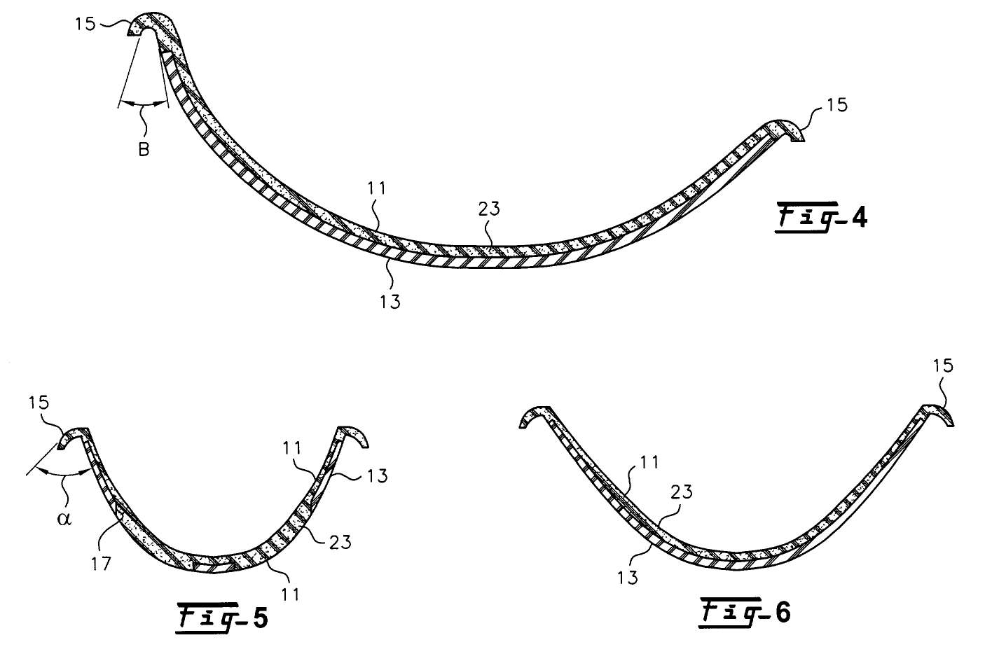

As shown in FIGS. 4-6, lip 15 is curved to permit the device to contact the user without presenting a stiff or sharp edge against the person The curve of lip 15 may range from more than about 45°, such as angle a in FIG. 5 to less than about 15°, such as angle β in FIG. 4. It is preferred that the angle the curved portion of lip 15 range from about 15° to about 60°.

To form the primary portion, a predetermined quantity of blowing agent is mixed with a thermoplastic material and the resultant mixture is then heated to the thermoplastic material's processing temperature range. The thermoplastic material then liquefies and the blowing agent decomposes into, inter alia, gaseous components to form gas pockets dispersed in the resultant thermoplastic composition The heated thermoplastic composition is then injection molded The cup is cooled and removed from the mold.

Upon initial use, the cup device is reheated to soften the polymer and to expand the gas pockets therein. The warm cup is then immediately placed in position and pressed gently into place, thus conforming the device to the personal dimensions of the user.

In the preferred embodiment, raw material pellets of ethylene vinyl acetate (EVA) are mixed with 1% to 3.5% powdered Celagen OT™, a product low in toxicity and manufactured by Uniroyal Chemical. The chemical composition of Celagen OT is p, p′-oxybis (benzenesulfonyl hydrazide) and it decomposes in the processing range of EVA to produce, inter alia, a gas mixture consisting of about 91% nitrogen gas and 9% water vapor. A molding machine then heats the mixture to approximately 320 degrees Fahrenheit as the mixture is injected into the cup mold. As the mixture is heated, the gas formed from the decomposition of the Celagen OT forms, and is trapped within, gas pockets, or bubbles, in the resultant thermoplastic composition. The exterior surfaces of the resulting cup are very smooth and thus do not encourage bacterial entrapment and growth.

Various other combinations of known thermoplastic materials and blowing agents may be used such that the selected blowing agents decompose to produce gas in the processing temperature range of the thermoplastic material. When used to form athletic cups or the like, the thermoplastic material, blowing agent and the resulting thermoplastic composition must be non-toxic and otherwise suitable for human contact.

The mold is cooled for approximately 30 seconds, separated, and injector pins release the cup from the mold. The cup thermoplastic composition contains approximately 10% gas pockets. The gas pockets are small collectively and in thicker areas the gas pockets are slightly larger since there was more space for expansion during injection and render an otherwise translucent EVA finished piece opaque. After the cup is removed from the mold, the gas trapped in the gas pockets exert a pressure greater than atmospheric pressure and tend to slightly puff the surrounding thermoplastic composition and the cup as a whole. After a few days, the gas pocket pressure equalizes against the surrounding thermoplastic composition and the puffing subsides.

The primary portion functions as a cushioning means and thus should have a degree of compression sufficient to prevent damage to the user's body when outside force is applied to the device.

The hard portion 13 functions as a cage or protective shell and is made from hard materials that functionally prevent the device from collapsing when subjected to impact, such as by a baseball or a hockey puck or the like. The preferred hard portion is made from rigid plastics. Most preferred is ABS, which is commercially available from most industrial plastics companies.

In order to function properly, the primary portion needs to provide a cushion to protect the person using the device. This is the function of the gas pockets. It is preferred that the primary portion have a hardness of less than about 60 on the Asker “C” scale. In a test device manufactured in accordance with the present invention using the preferred EVA polymer and foaming agent described above, the primary portion had an Asker “C” scale reading of 48.

The hard portion has to resist deformation to protect the user, and should have a hardness of at least 70 on the Asker “D” scale. In the same test device noted above, the hard portion was molded from ABS and had an Asker “D” scale hardness of 76.

A Hardness Tester is an instrument to quantify the sense of hardness or softness we experience by physical method. A spring load is pressed through a small indentor against a specimen surface to deform, thus providing a measure of ‘hardness corresponding to the depth of indentation below the specimen surface where the reaction of the specimen and the spring load reach equilibrium.

The Asker Hardness Tester is a well known product used for testing hardness and is available from a number of instrument supply companies. Preferred hardness testers are the ASTM D2240-specified Type D Durometer (ASKER Model D) for hard rubber and the ASKER Model C for soft rubber or flexible cellular materials.

Other testing devices may be employed to evaluate materials for use with the present invention, and the degree of cushion for the primary portion and the degree of rigidity for the hard portion are relative and can be adjusted, provided that the user is protected. The device of this invention has been tested in the laboratory and in use by athletes in a controlled environment to evaluate the efficacy of the invention. The tests have shown the device to provide excellent protection and comfort when used.

While particular embodiments of the present invention have been illustrated and described, it is not intended to limit the invention, except as defined by the following claims.

1. An athletic cup having a shape generally designed to have the narrow portion fit between the legs of the user while the upper or wider portion extends up over the area of the body being protected, comprising:
a primary portion formed from a resilient thermoplastic composition having a quantity of gas pockets dispersed therein; and
a hard portion bonded to said primary portion and forming an outer segment having a high degree of rigidity;
said primary portion also including a peripheral lip having a degree of flexibility for contact with the user.

2. The device of claim 1, wherein said hard portion includes open interstices and said primary portion extends into said interstices in said hard portion.

3. The device of claim 1, wherein said gas pockets are formed by heating of a mixture of a blowing agent and a resilient thermoplastic.

4. The device of claim 3, wherein said thermoplastic is ethylene vinyl acetate.

5. The device of claim 4, wherein said blowing agent is p, p′-oxybis (benzenesulfonyl hydrazide).

6. The device of claim 1, wherein said primary portion has a hardness of less than 60 on the Asker “C” scale.

7. The device of claim 1, wherein said hard portion has a hardness of at least 70 on the Asker D scale.

8. The device of claim 1, wherein said hard portion is ABS.

9. The device of claim 1, wherein said peripheral lip includes an arc having between about 15° and 60° of curve from the plane of said primary portion at said lip.

10. The device of claim 9, wherein said arc is between about 30° and 45° of curve from the plane of said primary portion.

11. An athletic cup having a shape generally designed to have the narrow portion fit between the legs of the user while the upper or wider portion extends up over the area of the body being protected, comprising:
primary portion means for providing a cushion resistance and formed from a resilient thermoplastic composition having a quantity of gas pockets dispersed therein; and
hard portion means for providing a crush resistance and bonded to said primary portion means and forming an outer segment having a high degree of rigidity;
said primary portion means also including a peripheral lip having a degree of flexibility for contact with the user.

12. The device of claim 11, wherein said hard portion means includes open interstices and said primary portion means extends into said interstices in said hard portion.

13. The device of claim 11, wherein said gas pockets are formed by heating of a mixture of a blowing agent and a resilient thermoplastic.

14. The device of claim 13, wherein said thermoplastic is ethylene vinyl acetate.

15. The device of claim 14, wherein said blowing agent is p, p′-oxybis (benzenesulfonyl hydrazide).

16. The device of claim 11, wherein said primary portion means has a hardness of less than 60 on the Asker “C” scale.

17. The device of claim 11, wherein said hard portion means has a hardness of at least 70 on the Asker D scale.

18. The device of claim 11, wherein said hard portion means is ABS.

19. The device of claim 1, wherein said peripheral lip includes an arc having between about 15° and 60° of curve from the plane of said primary portion at said lip.

20. The device of claim 19, wherein said arc is between about 30° and 45° of curve from the plane of said primary portion.
